(19) United States
(12) Patent Application Publication
(10) Pub. No.: US 2010/0056904 A1
(43) Pub. Date: Mar. 4, 2010

(54) IMAGE GUIDED INTERVENTION

(76) Inventors: John K. Saunders, Winnipeg (CA); Meir Dahan, Winnipeg (CA); Mauricio Ede, Winnipeg (CA); Steven Robbins, Winnipeg (CA)

Correspondence Address: ADE & COMPANY INC., 2157 Henderson Highway, WINNIPEG, MB, R2G1P9, CA

(21) Appl. No.: 12/202,795

(22) Filed: Sep. 2, 2008

(51) Int. Cl. A61B 5/05

(52) U.S. Cl. 600/424

(57) ABSTRACT

Image guidance on a computer screen in the patient's vascular system with reduced use of toxic contrast agent and X-ray radiation is obtained by providing anatomical and functional images obtained preferably from an MR Imaging system. A fibre optic device which uses strain measurement to provide an image of the shape and location of the fiber is used to provide spatial information of the elongate guide member as it is pushed through the vascular system with this spatial information being registered to the image of the vascular system by registering the location of the fiber/guide in the image prior to insertion using markers visible in the MR image.

This invention relates to a method for use in guiding a treatment device from an insertion site to a treatment site in a patient.

### BACKGROUND OF THE INVENTION

A number of procedures have been suggested in the past for treating disease conditions involving the narrowing or obstruction of the lumen of an artery. This condition, generally referred to as a lesion, is found in patients suffering from atherosclerosis, and can manifest itself as partial or total. The occlusions can be found at various locations in the arterial system, including the aorta, the coronary arteries, the carotid arteries and the peripheral arteries.

In the past, coronary artery occlusions have traditionally been treated by performing coronary bypass surgery, wherein in most cases, a segment of the patient's saphenous vein is taken from the patient's leg and is grafted onto the affected artery at points upstream and downstream of the occluded segment. While bypass surgery can provide dramatic relief, it involves major open chest surgery and typically a long period of convalescence.

In recent years less invasive procedures have been adopted for the treatment of arterial abnormalities. These procedures typically involve the use a catheter which is introduced into a major artery through a small arterial opening in the patient's body and is advanced into the area of the stenosis.

Popular prior art minimally invasive procedures include percutaneous transluminal coronary angioplasty, directional coronary atherectomy and endovascular stenting. Percutaneous transluminal coronary angioplasty typically involves the use of a balloon to mechanically dilate the stenosis. In carrying out this procedure, a steerable elongated guide wire is introduced into an arterial opening and advanced under x-ray fluoroscopy guidance into the stenosed artery and past the lesion. This done, a balloon catheter is advanced over the elongated guide wire until it is positioned across the stenotic area. The balloon is then inflated.

A somewhat similar prior art procedure, known as stenting, involves the use of a very small wire mesh, known as a stent, which is fitted over an inflatable balloon and is then positioned across the stenotic segment of the artery. When the stent is in the proper position, the balloon is inflated, dilating the stent and forcing it against the artery wall.

It is, of course, apparent that over-the-wire catheters cannot be positioned adjacent the stenosis until the elongate guide wire has been advanced across the stenosed area. In those instances where the artery is occluded, the surgeon may have greater difficulty in guiding the elongated guide wire through the occluded area. Thus, without some type of guidance system, the elongate guide member might undesirably impinge on and possibly perforate or otherwise damage the artery wall.

In light of the foregoing, there has been a long-felt need to provide a reliable guidance system for guiding a catheter through the occlusion. One prior art guidance system which has been used in conjunction with coronary catheterization involves bi-plane fluoroscopy, wherein the interventionist observes two 2-dimensional, real-time X-ray images acquired from different angles. However, bi-plane fluoroscopy has been proven to be somewhat costly, unreliable and slow.

Optical systems have also been disclosed for imaging an occlusion through a specially designed catheter positioned within the artery. One such system is Optical Coherence Tomography (OCT). In this system, a beam of light carried by an optical fiber illuminates the artery interior and light reflected back into the fiber from features inside the artery is correlated with the emitted light to capture the depth as well as the angular separation of those features. The features are displayed graphically in two or three dimensions through the use of a suitably programmed computer. Examples of such processing are given in U.S. Pat. No. 5,459,570 issued to Swanson.

Another prior art guidance system is disclosed in U.S. Pat. No. 6,010,449 issued to Selmon, et al. This patent discloses an intravascular catheter system that includes a steering apparatus, an imaging member and a therapeutic element within a multi-lumen catheter shaft. In one embodiment of the intravascular catheter system, a rotatable imaging shaft is disposed within the catheter shaft. The imaging shaft contains an optical fiber, which is connected to external optical instruments. At the distal end of the imaging shaft, the optical fiber conducts light from the instruments to illuminate the environment inside the artery and receives optical radiation returned from the environment. The imaging shaft is turned by an external motor encoder, which also measures the rotation of the shaft. As the imaging shaft rotates, the optical beam sweeps circumferentially about the longitudinal axis of the imaging shaft at a fixed angle from the longitudinal axis of the imaging shaft, illuminating different portions of the environment within the artery. The instruments correlate the emitted and received optical data with the rotational data to display an image of the interior of the artery.

Another technology for use in catheter guidance systems is Optical Coherence Reflectometry (OCR). The basic concepts of this technology have been well documented (see for example an article by Mandel L. Wolf entitled “Optical Coherence and Quantum Optics” published in the Cambridge University Press (1995)). In the practice of the OCR technology, a light source is divided into two beams, a reference arm and a sample arm. The light in the reference arm is reflected at a determinable path length. Light in the sample is also reflected or scattered by the material present in the sample. The reflections and back-scattered light are combined at an optic coupler, and if the path lengths of the two arms are within the coherence length of the light, the light will re-correlate or interfere with one another. The detector measures the interference intensity. Since the reference path length is known and adjustable, the intensity profile of scattered light from a sample can be determined as a function of the reference arm path length.

U.S. Pat. No. 6,451,009 issued to Dasilva, et al, discloses an optical coherence domain reflectometry (OCDR) guided laser ablation device. The Dasilva, et al, device includes a multimode laser ablation fiber that is surrounded by one or more single mode optical fibers that are used to image in the vicinity of the laser ablation area to prevent tissue damage. The laser ablation device is combined with an OCDR unit and with a control unit which initializes the OCDR unit and a high power laser of the ablation device. Data from the OCDR unit is analyzed by the control unit and is used to control the high power laser. The OCDR images up to about 3 mm ahead of the ablation surface to enable a user to see sensitive tissue such as a nerve or artery before damaging it by the laser.

A commercially available, prior art catheter system using the OCR technology is sold by IntraLuminal Therapeutics of Carlsbad, Calif. under the name and style “SAFE-STEER”. The IntraLuminal Therapeutics apparatus comprises an optical elongate guide member with an optical fiber integrated into it. The apparatus also includes an optical coherence reflectometry system which comprises an optical interferometer, a demodulation computer unit and monitor. In one form of the apparatus a single mode fiber with a polyimide jacket is used for the optics. The proximal portion of the elongate guide member is made up of commercially available hypodermic tubing that serves as a conduit for the fiber. In operation, the back-scattered light is analyzed through the low coherence interferometer producing a signal that is displayed and periodically updated on an OCR monitor.

Still another commercially available, prior art catheter system using radio frequency technology is sold by IntraLuminal Therapeutics of Carlsbad, Calif. under the name and style “SAFE-CROSS.” The Safe-Cross system was developed to effectively cross and re-canalize total occlusions and, according to the manufacturer, comprises a marriage of the OCR technology and controlled Radio Frequency (RF) energy to facilitate guidance through the occlusion.

There are three primary instruments routinely used in catheter insertion procedures. First, Michelson interferometers of various types are used to differentiate between plaque and arterial walls, and to do so with physical resolution in the range of 10 microns. Michelson interferometers provide the ability to see and navigate through a total occlusion. Second, Diffuse Reflectance Near Infrared Spectroscopy (DRNIRS), often with regard to multiple wavelengths, is effective at differentiating and identifying a wide variety of substances, including hundreds of plasma constituents, such as glucose, calcified plaque, vulnerable plaque, total protein, human metalloproteins, creatinine, uric acid, triglycerides, uric acid, urea, etc. DRNIRS interferometry provides the capability to detect and determine materials without actually contacting or touching them. The substances are distinguished by the characteristic absorption and reflectance of specific wavelengths of light, typically between 300 and 2200 nanometers. Third, excimer lasers typically use a very short pulse, less than 1 microsecond, normally about 100 nanoseconds, and could be operated together with both types of interferometry in duty cycles as high as hundreds of hertz.

Examples of optical systems which utilize a fiber to obtain image information from the end of the fiber within the blood vessel are shown in US 2005/0171437 of Carberry published Aug. 4, 2005 and US 2006/0229591 of Lee published Oct. 12, 2006.

With Magnetic Resonance Imaging, a high field magnet, typically superconducting, is arranged in a torus configuration and with the patient lying down inside the magnet the magnetic field allows a pulsed and sequenced magnetic and EM field to probe the body to produce soft tissue images, which allow the trained radiologist to determine with high probability the anatomy of the patient. MRI is sometimes performed using contrast agents to provide even better contrast between different soft tissue types. MRI techniques are very good at imaging soft tissues and detecting the anatomical function of the blood vessels.

In U.S. Pat. No. 5,735,278 (Hoult et al) issued Apr. 7, 1998, is disclosed a medical procedure where a magnet is movable relative to a patient and relative to other components of the system. The moving magnet system allows intra-operative MRI imaging to occur more easily in neurosurgery patients, and has additional applications for liver, breast, spine and cardiac surgery patients. The disclosure of this issued patent is incorporated herein by reference.

In Published PCT Application WO/07/147233A1 of the present Applicants published Dec. 27, 2007 and entitled ROTATABLE INTEGRATED SCANNER FOR DIAGNOSTIC AND SURGICAL IMAGING APPLICATIONS is disclosed an improvement to the above patent in which an additional rotational movement of the magnet is allowed. The disclosure of this published application is incorporated herein by reference.

Interventional arterial treatment requires accurate determination of where the catheter or other device is in the arterial system. This is mainly performed presently by X-Ray fluoroscopy where X-Ray imaging determines the position of elongate guide members or catheters as they are pushed through the arterial system. The patient's arteries are detected by use of contrast reagent which is based upon iodine which is radio opaque. The analysis of the angiograms provides the interventionist with the arterial system and also the location of any devices such as elongate guide members, catheters etc. The fluoroscopy technology provides information on the arteries and the position of devices in the arteries but does not provide any information on surrounding anatomy. Two additional drawbacks of this technology are the toxicity of the contrast reagent and the effect of the X-ray radiation on human tissue.

Recently a technique has become available, as disclosed in US 2007/0065077 of Childers assigned to Luna Innovations Inc and published Mar. 22, 2007, for a fiber optic position and shape sensing device. The device has either at least two single core optical fibers or a multi-core optical fiber having at least two fiber cores. In either case, the fiber cores are spaced apart such that mode coupling between the fiber cores is minimized. An array of fiber Bragg gratings are disposed within each fiber core and a frequency domain reflectometer is positioned to receive light from the optical fiber. In use, the device is affixed to an object which causes distortion of the device. Strain on the optical fiber is measured and the strain measurements correlated to local bend measurements. Local bend measurements are integrated to determine position and/or shape of the object. The disclosure of this published application is incorporated herein by reference.

Luna have entered into a relationship with Intuitive Surgical, manufacturers of the Da Vinci Surgical Robot for use of the above position sensing technique in sensing the position of the robot.

A prior U.S. Pat. No. 5,563,967 of Haake issued Oct. 8, 1996 to McDonnell Douglas discloses a fiber optic sensor which uses similar principles to the Luna device for detecting strain and other parameters. The disclosure of this issued patent is also incorporated herein by reference.

### SUMMARY OF THE INVENTION

According to the invention there is provided a method for use in guiding a treatment device along a path from an insertion site to a treatment site in a patient: comprising:

placing the patient in an imaging system and operating the imaging system to generate at least one image of the path within the patient showing a path through the blood vessels from the insertion site to the treatment site;

providing an optical fiber system having an optical fiber member;

providing an elongate guide member attached to or integral with or defined by the optical fiber member arranged such that the elongate guide member can be manipulated along the path from the insertion site to the treatment site;

the optical fiber member having components therein such that the optical fiber system can determine the position and shape of at least a portion of the fiber within a coordinate system by analysis of light received from the optical fiber member in response to light transmitted into the optical fiber member;

generating a registration of the coordinate system of the optical fiber system with the image;

and guiding the elongate guide member to the treatment site using data from the optical fiber system while applying the data onto said at least one image of the path using the registration.

The method is particularly useful in vascular treatment so that the path is through blood vessels of the patient.

The term “elongate guide member” is not intended to limit the feature to any particular structure or material and the feature may include a conventional metal wire as part of the structure or it may not. The fiber may form the whole of the elongate guide member or may be supplemented by additional structure to provide required mechanical properties. The elongate guide member may be in the form of a catheter or other usable tool within the vessel or may be just a carrier for the tool to be inserted later. The elongate guide member may be hollow or tubular or may form a core.

The optical fiber member may include a single fiber or a plurality of individual fibers.

Preferably the imaging system is moved away from the patient during the guiding such that the elongate guide member is guided using the data from the optical fiber system which is applied onto the image previously obtained. However it will be appreciated that repeated imaging may occur to update information or to provide supplemental information if required.

Preferably the registration of the coordinate system of the optical fiber system with the image is generated by marking within the image a plurality of known points on the optical fiber member. This is preferably done where the known points on the fiber member carry markers visible on the imaging system.

In one embodiment, the imaging system is a single plane X-ray system or a bi-plane X-ray system. In known manner this imaging system can generate a visible image or a series of images of the arterial system from the insertion site to the treatment site.

Alternatively the imaging system can be a CT system.

In both of these cases, it is preferred that the initial imaging system be moved away after the initial imaging is complete and a subsequent imaging effected using a movable MRI magnet system of the type described in the above documents of the present Applicants.

Alternatively the imaging system can be a Magnetic Resonance Imaging system arranged to effect in an initial step a Magnetic Resonance Angiography. In this arrangement preferably the Magnetic Resonance Imaging system includes a magnet which is movable away from a patient support so as to be spaced away from the patient during the guiding. In this way the guiding is effected wholly by the optical fiber system without the magnet providing any inference with the guiding action.

Preferably the magnet is returned to the patient after the guiding for Magnetic Resonance Imaging of the patient after the treatment is completed so that the effect of the treatment is monitored using the effective functional imaging obtainable in MRI while the treatment modality is still available at the treatment site for a repeat if the treatment is found to be unsatisfactory.

Preferably the known points carry markers of a material visible on the Magnetic Resonance Imaging system such as small spherical balls filled with aqueous solution of milli molar paramagnetic salts such as MnCl₂.

Preferably said at least one image comprises a series of MR images in slices and preferably the series of images in slices are combined into a three-dimensional image onto which the position and shape of the optical fiber member is applied using the previously obtained registration.

Preferably the guiding is effected using solely the data from the optical fiber system applied onto said at least one image of the blood vessels using the registration, without the necessity for any other imaging modality such as visual optical systems or tissue analysis as explained hereinbefore. However this technique may also be used in conjunction with other known techniques. Thus preferably the image taken is of the blood vessels taken without any contrast reagent.

Preferably the optical fiber member includes an array of fiber Bragg gratings for measuring strain on the optical fiber member from which local bend measurements can be determined for analyzing the position and shape of the optical fiber member. However other analysis methods may be used to determine the shape and/or position of the optical fiber member.

A drawback found in certain of the prior art OCR optical fiber elongate guide member systems resides in the fact that the optical fiber elongate guide member tends to be substantially more difficult to navigate through the artery passageway than the catheters embodying more conventional metal elongate guide members such as are used in stent delivery and like procedures. The apparatus described herein can include a catheter system that includes both an optical fiber for use in expeditiously guiding the catheter and a conventional metal elongate guide member for use in navigating the catheter through the artery passageway.

The arrangement described herein provides image guidance on a computer screen in the patient's vascular system with minimal use of toxic contrast agent and X-ray radiation and will provide anatomical and functional images. The fibre optic device provides spatial information of the elongate guide member as it is pushed through the vascular system and this spatial information is registered to the vascular system and anatomical images.

The workflow will be the following;

1. Image the vascular and the anatomy with MRI, MRA or X-Ray Angiography or CT.

2. Insert the fibre optic cable with MRI and/or X-Ray markers.

3. Register the MRI (MRA, Angio, CT) data to the fibre optic data.

4. Place an MRI marker at the tip of the fibre optic cable where the position of the tip can be determined using the optical fiber system. This is used to guide MRI imaging planes with respect to the tip of the cable or the tip of the catheter.

5. The insertion and movement of the fibre optic cable with the attached catheter or guide wire is guided on the computer screen using the location fiber optic location system which locates the position of the fibre optic cable. The registration of the fiber optic location system in the image is similar to how the neurosurgeons instrument with the IR reflecting balls described the movement of the tip of the instrument in the patient's brain.

6. The patient is imaged in a rapid CT Angiogram or diagnostic fluoroscopy to ascertain the number and position of artery stenosis followed by a rapid MRI (perfusion, function viability) to image the heart and determine the exact course of treatment.

7. The fibre optic cable having been registered during the above MRI measurements is used to guide the treatment, for example the placing of a stent.

8. After the placement is complete a further MRI is used to determine that the treatment has had the desired effect.

9. In some cases, a CT Angiogram or a diagnostic fluoroscopy exam may be used to guide stent positioning and prove that stent positioning has been obtained as desired.

10. In Electro Physiology (EP) applications, the fibre optic system may be used to guide the catheter to the desired position and then MRI may be used to guide the treatment, for example cryogenic therapy or thermal therapy including focussed ultra sound (FUS), and to verify that the treatment had the desired effect. The aim of the treatment is to ablate certain electrical paths which result in undesired electrical signal be transmitted (attrial fibrillation is a well known example and for this application ablation is conducted around the pulmonary veins).

The technology can also be used for percutaneous valve replacements where the insertion site is through the skin and into a suitable blood vessel normally in the groin or the neck of the patient.

The technology is also very useful for the treatment of stroke. Unlike the coronary arteries which are very difficult to image with MRI or MRA, the main arteries in the brain can be imaged quickly and precisely with MRA and in this case the method can use MRI as the sole imaging process with the laser fibre optic technology properly registered to the image. The clot causing the stroke can be eliminated by guiding a clot busting drug directly to the clot or by removing the clot with a mechanical clot remover such as that developed and patented by Concentric Medical known in the industry as the MERCI device.

In summary, the arrangement described herein includes the ability to obtain spatial information of a guidance device using the fiber optic system which includes a laser and the fibre optics, the ability to include this spatial information on CT, MRI and diagnostic images by registration in the image space of the fibre optic space, the ability to navigate in image space in a similar fashion to optically guided surgical navigation, the ability to fuse image from different imaging modalities and the ability to use MRI to guide treatment and verify treatment success.

This invention permits the treatment of a number of cardiac diseases with a minimal or zero use of X-Ray irradiation and nephro-toxic contrast reagent. The image guidance can be performed simultaneously with MRI or CT imaging. It can be used to guide slice selection in MRI imaging so that the required planes are always obtained. The use of fibre optic system eliminates the line of sight challenge which influences image guidance based on optical camera methods.

Guidance in the vascular system is conventionally carried out using fluoroscopy which has been used for years but this does not give any anatomical information. In the present arrangement, the simultaneous guidance within the vascular with anatomical information is obtained using no radiation and no adverse contrast reagents.

As set out hereinbefore, all of the imaging can be carried out using an MRI system to generate initially an MR Angiogram, for use in the guidance of the elongate guide member through the blood vessels from the insertion site to the treatment site using the optical fiber system, and then subsequently to carry out the imaging of the treatment site using MRI.

Alternatively the optical fiber system which can determine the position and shape of at least a portion of the fiber can initially be used in an X-ray angiography system which is mono-plane or bi-plane and then subsequently the further imaging is effected using the MRI system.

### BRIEF DESCRIPTION OF THE DRAWINGS

FIG. 1 is a schematic side elevational view of an MRI system and an fiber optical locating system used in conjunction in accordance with the present invention.

FIG. 2 is a schematic illustration of the positioning of the guide member and optical fiber member within a blood vessel of a patient.

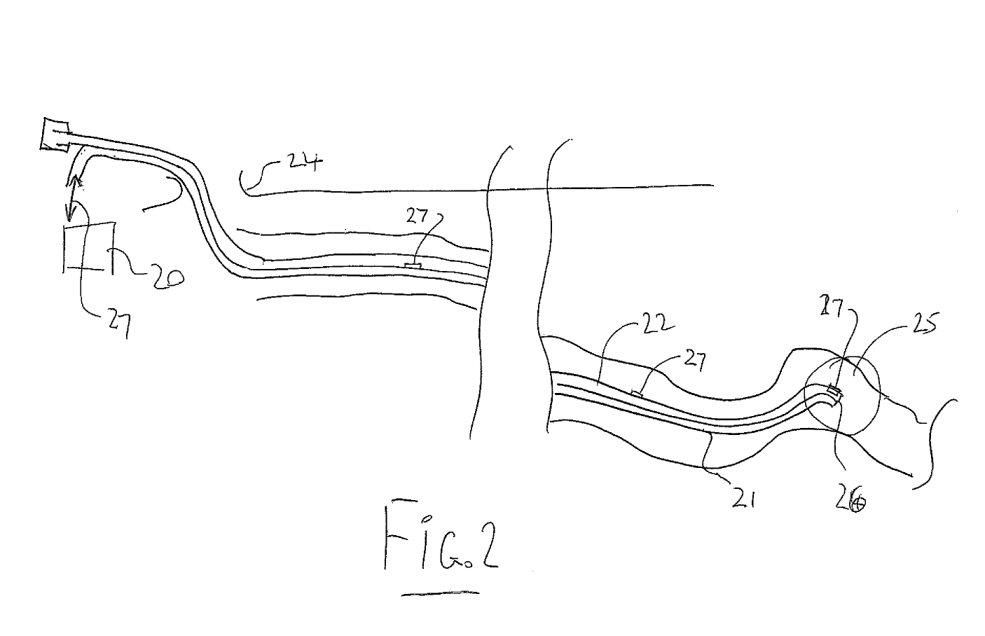

### DETAILED DESCRIPTION

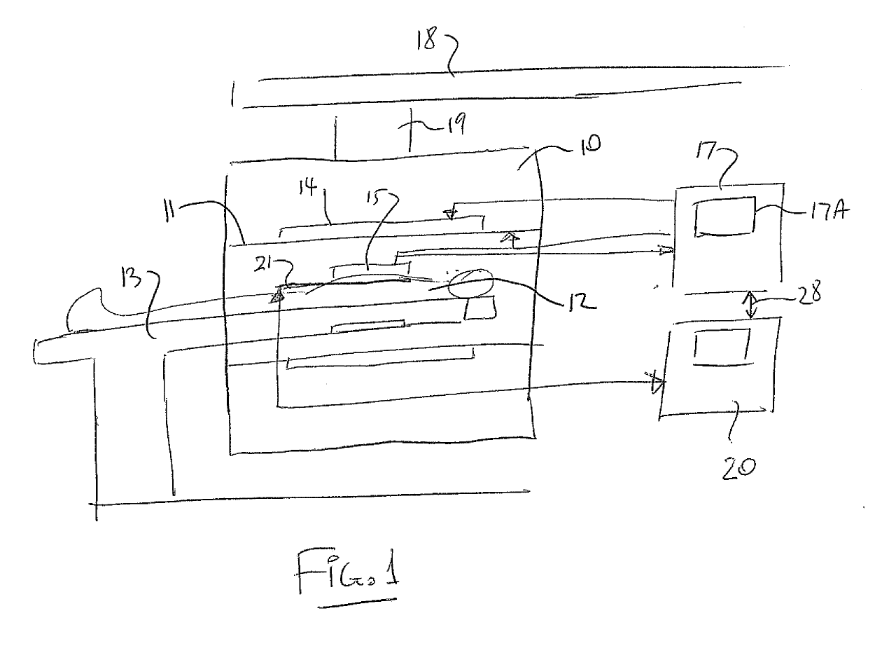

In FIG. 1 is shown schematically a magnetic resonance imaging system which includes a magnet 10 having a bore 11 into which a patient 12 can be inserted on a patient table 13. The system further includes an RF transmit body coil 14 which generates a RF field within the bore.

The system further includes a receive coil system generally indicated at 15 which is located at the isocenter within the bore and receives signals generated from the human body in conventional manner. A RF control system 17 acts to control the transmit body coil 14 and to receive the signals from the receive coil 15. The magnet is carried on a rail system 18 by a support 19 so that the magnet and associated operating components can be moved into place at the patient on the table and can be removed to allow the surgeon to carry out the necessary actions on the patient.

Further details of this arrangement are described in the above U.S. Pat. No. 5,735,278 (Hoult et al) and the above PCT Application WO/07/147,233A1 of the present Assignees, the disclosures of which are incorporated herein y reference.

The control system 17 is shown only schematically and is used in well known manner to generate an image to be displayed to the medical personnel on a display schematically indicated at 17A.

There is also provided an optical fiber detection system 20 having an optical fiber member 21 which is connected to or carried by an elongate guide member 22 such that the elongate guide member can be manipulated through the blood vessels 23 from the insertion site 24 to the treatment site 25. The optical fiber member is of the type previously described and explained in detail in US 2007/0065077 of Childers assigned to Luna Innovations Inc. to which reference may be made for further details. The optical fiber member includes Bragg gratings for measuring strain on the optical fiber member from which local bend measurements can be determined for analyzing the position and shape of the optical fiber member. Thus the optical fiber system can determine the position and shape generally the whole working length of the fiber 21 from the tip 26 at the treatment site to the insertion site, that is at least a portion of the optical fiber member within a co-ordinate system determined by the optical fiber system by analysis of light received from the optical fiber member in response to light transmitted into the optical fiber member as indicated at 27. The shape and position of the fiber member in the coordinate system is displayed on a display 20A of the system 20.

The fiber or the guide carry markers 27 at spaced positions along the fiber which are visible in the MR imaging system so as to be displayed on the image 17A.

Software provides communication between the images of the system 20 and the imaging system 17 as indicated at 28. This allows both systems to generate an image of the location of the fiber/guide so that the images can be registered for viewing as a common image on a viewing system visible by the medical personnel.

In operation, the vascular system and the anatomy of the patient are image using the MRI or MRA system to obtain at least one image of the blood vessels of the patient from the insertion site to the treatment site. The image of the blood vessels can be taken without any contrast reagent.

The fibre optic member 21 is located in the image with the vascular system and the anatomy of patient using the MRI markers 27.

The fibre optic data obtained by the system 17 from the fiber is registered with the MRI or MRA data to overly the image of the fiber/guide on the patient. The markers preferably include an MRI marker at the tip of the fibre optic member so that the position of the tip can be determined using the optical fiber system. This is used to guide the selection of MRI imaging planes with respect to the tip of the fiber member between the insertion site 24 and the treatment site 25 so that the path of the fiber/guide can be tracked on a composite MR image obtained from those selected slices.

With the magnet of the imaging system removed so that no further real time imaging of the fiber/guide is possible in the MR system, the insertion and movement of the fibre/guide is guided on the computer screen using the fiber optic location system which locates the position of the fibre/guide and particularly the tip 26. Thus the guiding is effected using solely the data from the optical fiber system applied onto said at least one image of the blood vessels using the registration. The registration of the fiber optic location system in the image is similar to how the neurosurgeons instrument with the IR reflecting balls describes the movement of the tip of the instrument in the patient's brain as outlined in U.S. Pat. No. 6,859,660 (Vilsmeier) issued Feb. 22, 2005 to BrainLab AG, the disclosure of which is incorporated herein by reference.

The fibre member having been registered during the above MRI measurements is used to guide the treatment, for example the placing of a stent using data from the optical fiber system while applying the data onto said at least one image of the blood vessels using the registration. After the placement is complete a further MR Image is obtained with the magnet returned to the imaging location and is used with to determine that the treatment has had the desired effect.

In some cases, a CT Angiogram or a diagnostic fluoroscopy exam may be used to guide stent positioning and prove that stent positioning has been obtained as desired.

1. A method for use in guiding a treatment device along a path from an insertion site to a treatment site in a patient: comprising:
placing the patient in an imaging system and operating the imaging system to generate at least one image of the path within the patient showing a path through the blood vessels from the insertion site to the treatment site;
providing an optical fiber system having an optical fiber member;
providing a elongate guide member arranged to be carried with the optical fiber member such that the elongate guide member can be manipulated from the insertion site to the treatment site;
the optical fiber member being arranged such that the optical fiber system can determine the position and shape of at least a portion of the optical fiber member within a coordinate system by analysis of light received from the optical fiber member in response to light transmitted into the optical fiber member;
generating a registration of the coordinate system of the optical fiber system with said at least one image of the path;
and guiding the elongate guide member to the treatment site using data from the optical fiber system while applying the data onto said at least one image of the path using the registration.

2. The method according to claim 1 wherein the imaging system is moved away from the patient during the guiding such that the elongate guide member is guided using the data from the optical fiber system which is applied onto said at least one image of the blood vessels previously obtained.

3. The method according to claim 1 wherein the registration of the coordinate system of the optical fiber system with said at least one image of the path is generated by marking within said at least one image a plurality of known points on the optical fiber member.

4. The method according to claim 3 wherein the known points carry markers visible on the imaging system.

5. The method according to claim 1 wherein the imaging system is a single plane X-ray system.

6. The method according to claim 1 wherein the imaging system is a bi-plane X-ray system.

7. The method according to claim 1 wherein the imaging system is a CT system.

8. The method according to claim 1 wherein the imaging system is a Magnetic Resonance imaging system arranged to effect Magnetic Resonance Angiography.

9. The method according to claim 8 wherein the Magnetic Resonance Imaging system includes a magnet which is movable away from a patient support so as to be spaced away from the patient during the guiding.

10. The method according to claim 8 wherein the magnet is returned to the patient after the guiding for Magnetic Resonance Imaging of the patient after treatment.

11. The method according to claim 8 wherein the known points carry markers of a material visible on the Magnetic Resonance Imaging system.

12. The method according to claim 1 wherein said at least one image comprises a series of images in slices.

13. The method according to claim 12 wherein said series of images in slices are combined into a three-dimensional image onto which the position and shape of the optical fiber member is applied.

14. The method according to claim 12 wherein data from the optical fiber system is used to guide slice selection in the imaging.

15. The method according to claim 1 wherein the guiding is effected using solely the data from the optical fiber system applied onto said at least one image of the path using the registration.

16. The method according to claim 1 wherein said at least one image of the path is taken without any contrast reagent.

17. The method according to claim 1 wherein the optical fiber member includes an array of fiber Bragg gratings for measuring strain on the optical fiber member from which local bend measurements can be determined for analyzing the position and shape of the optical fiber member.

18. The method according to claim 1 wherein the path extends through blood vessels of the patient.
